(19) United States
(12) Patent Application Publication
(10) Pub. No.: US 2014/0378911 A1
(43) Pub. Date: Dec. 25, 2014

(54) ELECTRONIC INJECTOR

(71) Applicant: Q-Med AB, Uppsala (SE)

(72) Inventors: Jonas Dolk, Bromma (SE); Hans Himbert, Uppsala (SE); Jonas Törnsten, Uppsala (SE); Max Blomqvist, Uppsala (SE)

(21) Appl. No.: 14/346,785

(22) PCT Filed: Sep. 28, 2012
(86) PCT No.: PCT/EP2012/069192

§ 371 Date: Mar. 24, 2014

(30) Foreign Application Priority Data
Sep. 28, 2011 (EP) 11183169.9

(51) Int. Cl. A61M 5/20

(52) U.S. Cl. CPC A61M 5/20; A61M 2005/2411
USPC 604/227

(57) ABSTRACT

An injection device is provided for delivering liquid compositions, such as viscous hyaluronic acid gels. The injection device includes a generally elongated housing having first and second ends. The housing is configured to receive an exchangeable cartridge having the liquid composition. The cartridge is received proximal to the first end of the housing. A drive mechanism is arranged within the housing and includes an electric motor and a power source, typically a rechargeable battery. The motor is coupled to a plunger rod which acts on the cartridge received in the housing to expel the liquid composition contained in the cartridge. A first actuation device for actuating the drive mechanism is provided on an upper side of the housing proximal to the first end of the housing, and a second actuation device for actuating the drive mechanism is provided at the second end of the housing.

### TECHNICAL FIELD

The present disclosure relates to an injection device for delivering liquid compositions, such as viscous gels of e.g., hyaluronic acid.

### BACKGROUND

WO2008/020023 discloses an injection device for injection of a predetermined quantity of a medicament. That injection device comprises a drive device for pushing a piston within a cartridge containing the medicament such that said quantity is expelled from the cartridge through an opening of a needle provided at one end of the cartridge. The drive device of that prior art document includes an energy accumulating member in the form of a spring which is wounded in advance by means of a tensioning knob. In order to expel the predetermined quantity, a user seizes the device similar to a pen and pushes a button provided near a front end of the device with his or her index finger. Even though that device has obvious advantages over normal handheld syringes, it has drawbacks concerning user ergonomics. For example, the construction of that device makes its rear end comparatively heavy which can be strenuous when the device is used for longer periods of time. Furthermore, there is always a risk that the accumulated energy will not suffice for the complete duration of the treatment which creates an unnecessary interruption of the treatment.

### SUMMARY

It is an object of the present disclosure to provide an improved injection device which reduces or eliminates the above mentioned and other drawbacks. This object and other objects are achieved by an injection device according to the embodiments discussed below. This object and other objects are also achieved by an injection system discussed herein. Thus, in accordance with an aspect of the present disclosure there is provided an injection device for delivering a liquid composition, comprising a generally elongated housing having first and second ends. The housing is adapted to receive an exchangeable cartridge comprising the liquid composition to be delivered proximal to said first end of said housing. A drive mechanism is arranged within the housing and comprises an electric motor and a power source. The motor is coupled to a plunger rod for acting on a plunger of a cartridge received in said housing such that the liquid composition contained in the cartridge can be expelled. In order to activate the drive mechanism, a first actuation means is provided on an upper side of the housing, proximal to said first end of the housing and a second actuation means is provided at said second end of the housing. The electric motor and the power source can be provided within the housing such that the centre of gravity of the injection device is located at an appropriate position for obtaining good ergonomics. Due to the fact that actuation means are provided at two different positions of the injection device, a user can hold the device in a position similar to how a pen is held but also in a position similar to how a traditional hand-held syringe is held. Especially the latter position is preferred among many users since they are used to work with traditional hand-held syringes in this way. Furthermore, by using an electric motor, the working operation of winding the energy accumulating member in the form of a spring is avoided. When using traditional hand held syringes, the injection rate can be hard to control at all times due to the high viscosity of hyaluronic acid gel typically used which requires the user to apply a high force on the plunger rod of such traditional syringe. A too high injection rate during injection of e.g., hyaluronic acid gel, may cause bruises and swelling. Further, injection of liquid compositions sometimes require a controlled injection of very small amounts which can be difficult to achieve with hand held syringes. The provision of an electric motor has the advantage that the injection speed, the rate at which the liquid composition is delivered, can be set to a preferred value, thus avoiding the above-mentioned problems.

In accordance with an embodiment of the injection device of the present disclosure, the power source comprises a rechargeable battery. A rechargeable battery require less space than the spring described in WO2008/020023 and can be obtained in different sizes and different shapes. This results in a possibility of obtaining a favourable centre of gravity since a rechargeable battery can be positioned within the housing in many ways and that considerations of the space available within the housing can be attended to.

In accordance with an embodiment of the injection device of the present disclosure, contact plates are provided on the housing through which the rechargeable battery can be connected to a charging station for recharging. This has the advantage that no male/female cable connections have to be provided on the injection device. Such connections are prone to cause loose contact, thus hampering the charging of the battery. Further, such connections are often difficult to clean leading to residues and corrosion thereby impairing hygiene as well as reliable connection between the device and charger.

In accordance with an embodiment of the injection device of the present disclosure, finger grips are provided on opposite first and second side walls of the elongated housing. The finger grips facilitate the operation of the injection device when it is held as a traditional hand-held syringe.

In accordance with an embodiment of the injection device of the present disclosure, the position of the finger grips on said first and second side walls of the elongated housing is adjustable. Adjustable finger grips allow for users to always find a comfortable grip no matter what hand size he or she has.

In accordance with an embodiment of the injection device of the present disclosure, the drive means is capable of exerting a force of up to about 100 N on the liquid composition contained in a cartridge received in the housing. The required force depends on a wide range of parameters, such as the type of liquid composition to be delivered, the resistance provided by tissue at the location of delivery and the dimensions of the injection system (needle gauge, plunger diameter etc.). Therefore, the drive means should be able to exert a force on the liquid composition within a wide range. Typically viscous gels of e.g., hyaluronic acid, require a force of about 5-50 N, and depending on the combination of substances to be injected and the needle that is used, 30-40 N might be preferable.

In accordance with an embodiment of the injection device of the present disclosure, the first and second actuation means comprise encapsulated switches. Such switches assure good hygiene since they are easy to keep clean.

In accordance with an embodiment of the injection device of the present disclosure, at least one of the first and second actuation means comprises tactile feedback means for providing, for example, indications to a user about the status of an ongoing injection. A tactile feedback means can be very useful in practice since it allows for a user to continuously receive information from the injection device.

In accordance with an embodiment of the injection device of the present disclosure, at least one of the first and second actuation means comprises a touch switch. A touch switch can be made to comply with even higher hygiene standards since it can be mounted within the housing of injection device and no moving parts or sealings are required.

In accordance with an embodiment of the injection device of the present disclosure, means are provided for obtaining and presenting information to a user pertaining to an inserted cartridge. This reduces the risk that a cartridge comprising the wrong type of liquid composition is introduced into the device, thereby enhancing patient safety.

In accordance with an embodiment of the injection device of the present disclosure, the housing is made from plastics. Plastics is convenient since it is easy and cost-effective to produce in any desired shape and it can be produced to comply with tough hygienic limits.

In accordance with an embodiment of the injection device of the present disclosure, parts of the outer surface of the housing are provided with grip enhancing surface coating. This is convenient in order to avoid that a user slips when using the injection device, possibly causing pain and distress to a patient.

In accordance with another aspect of the present disclosure, an injection system for delivering a liquid composition is provided, comprising an injection device according to any of the preceding claims, an exchangeable cartridge containing the liquid composition, and an injection needle attached to said cartridge, wherein said cartridge is held by said housing.

In accordance with an embodiment of the injection system of the present disclosure, the exchangeable cartridge comprises finger grips. This facilitates for a user to hold the injection system in a syringe-like position. It would also be conceivable to provide exchangeable cartridges with varying finger grip positions, i.e. adapted to different hand sizes. In this way, a user can choose an exchangeable cartridge where the finger grips are positioned at a comfortable distance from the second end of the housing.

In accordance with an embodiment of the injection system of the present disclosure, the diameter of the injection needle ranges from 7 to 34 gauge.

Further objects and advantages of the present disclosure will be discussed below by means of exemplifying embodiments. These and other features, aspects and advantages of the present disclosure will be more fully understood when considered with respect to the following detailed description, appended claims and accompanying drawings.

### BRIEF DESCRIPTION OF THE DRAWINGS

Exemplary embodiments will now be described in more detail and with reference to the appended drawings in which:

FIGS. 1 and 2 are schematic perspective views of an embodiment of the injection system.

FIG. 2a is a schematic perspective views of an embodiment of the injection system when held by a user in a pen-like position.

FIG. 2b is a schematic perspective views of an embodiment of the injection system when held by a user in a syringe-like position.

FIG. 2c is a schematic perspective view of an embodiment of the injection system when held in an alternative position.

FIG. 2d is a schematic perspective view of an embodiment of the injection system when held in an alternative position.

FIG. 3 is a schematic perspective view of a number of injection systems positioned in a rack.

FIGS. 4a through 4c illustrate schematic perspective views of a second embodiment of the injection system.

### DESCRIPTION OF PREFERRED EMBODIMENTS

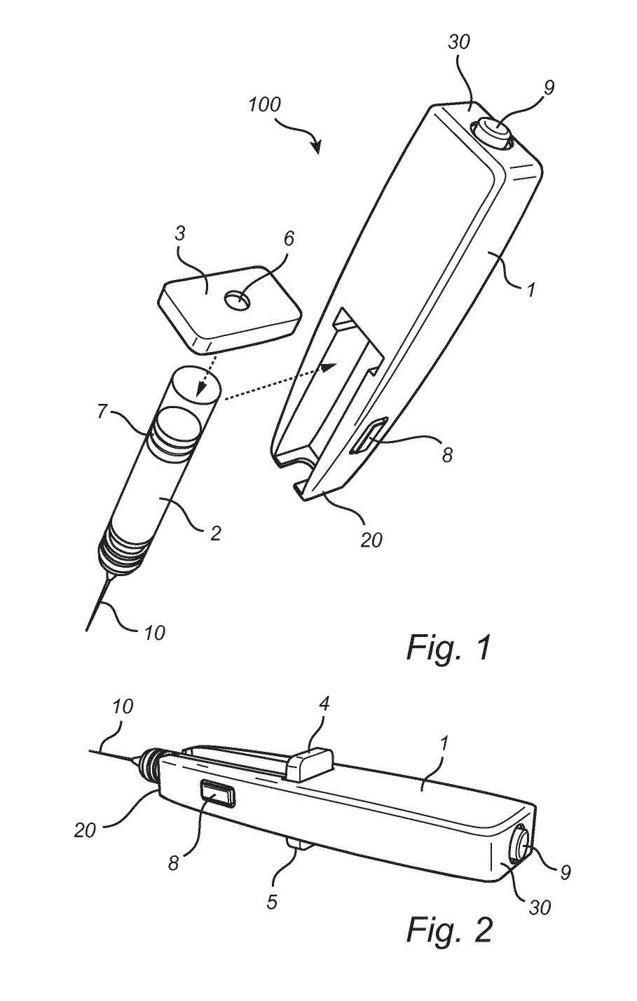

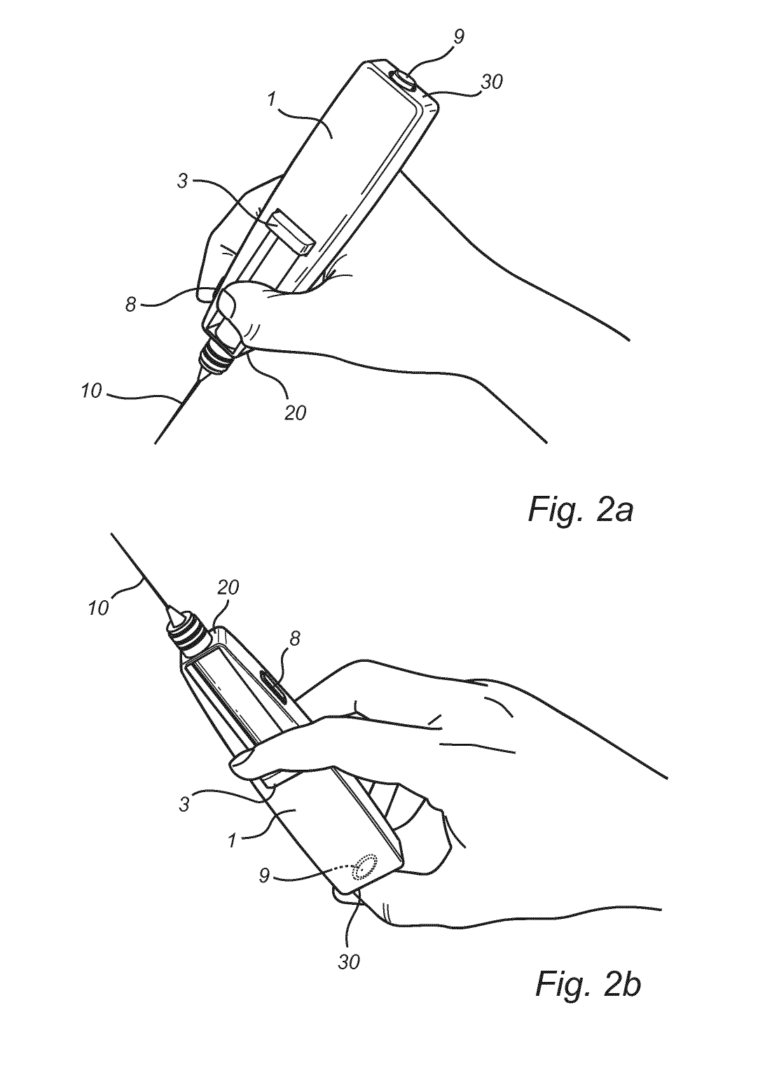

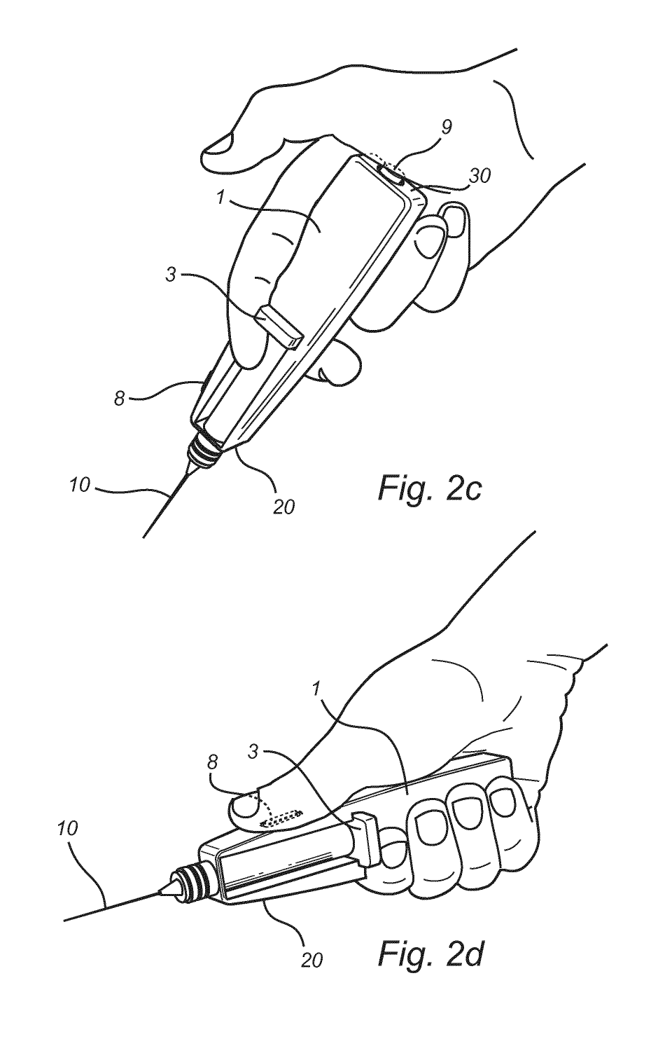

In a first embodiment of an injection system according to the present disclosure, as shown in FIGS. 1 and 2, an injection system 100 comprises a housing 1 made from thermoplastic and a cartridge 2. As used herein, the term “cartridge” encompasses all types of containers suitable for injectable liquid compositions, including gels. Further, the cartridge may comprise a rigidly mounted needle or a needle may be arranged, e.g., by threading, at one end of the cartridge. Also, the term cartridge may as used herein be considered to comprises a carrier structure or similar. As shown in FIG. 1, the cartridge 2 can be inserted and firmly held within the housing 1. In this embodiment, the cartridge 2 is provided with a separate plate 3 providing finger grips 4 and 5. The plate 3 can be slipped onto the cartridge 2 and they may be attached to each other by means of a snap-connection in order to ensure a reliable coupling between the two parts. Reliable coupling between the cartridge 2 and the plate 3 can also be obtained by providing a press fit between the parts. The plate 3 and the cartridge 2 may of course also be integrally formed. It is also possible to include the finger grips in the housing 1. This has the advantage that finger grips are provided to a user whether finger grips are provided on the cartridge 2 or not since the provision of finger grips on one of the part does not exclude the provision of finger grips on the other. The cartridge 2 together with the plate 3 is inserted in the housing 1 as shown in FIG. 2 and is being firmly held in this position by means of a snap-connection or a press fit or even a separate locking member. When the cartridge 2 is correctly positioned in the housing, a plunger rod, not shown in the figures, driven by a motor of the injection device 100, also not shown in the figures, extends through the opening 6 in the plate 3 into the cartridge 2, thus engaging with the plunger 7 provided within the cartridge 2. In one embodiment, the plunger rod is driven by the motor through a threaded connection, see also further below, and consequently it performs its rectilinear motion under rotation. In this case, a spacer piece can be provided at the leading end of the plunger rod to reduce friction between plunger rod and plunger. The spacer piece is preferably rotatably journalled at the end of the plunger rod to avoid that the rotational motion acts upon the plunger. The spacer piece is preferably manufactured from a low friction material to reduce losses due to friction between itself and the plunger rod. The plunger rod can be driven forwards by a user by pushing one of the actuation means 8, 9, shown in the figures as encapsulated switches. It should be noted in this matter, that the actuation means are by no means restricted to encapsulated switches. The skilled person readily realizes that many other types of switches may be applied, such as touch switches and membrane switches. Tactile switches which respond differently depending on the force applied to it by a user are also conceivable. For example, a high depressing force on the switch trigger could cause the injection device 100 to expel the liquid composition at a higher rate compared to when a lower force is applied. The switches can also utilize haptic technology, i.e. taking advantage of a user's sense of touch by applying forces, vibrations, and/or motions to the user, such that feedback is provided to a user through the switch. It would thus be possible to have feedback through the switch regarding a number of parameters concerning the injection process, for example the expelling rate of the liquid composition and the resistance against which the liquid composition is expelled. It would also be possible to inform a user by haptics that the amount of liquid composition within the cartridge 2 is drawing towards an end or that there are compatibility problems between the cartridge 2 and the housing 1 due to faulty installation or due to installation of a cartridge 2 which is not intended for use with the injection device 100. Instead of haptics, or as a complement thereto, information may also be displayed on an LCD-screen, not shown in the figures, provided on the housing 1. An easy and yet reliable way of providing information to a user is to use one or more LED-lights on the housing that indicate e.g., proper function of the device; injection taking place; what type of injection is taking place (e.g., intermittent or continuous injection); failure of the device; re-charging in process; low battery indication; and many more. Sound indications are also imaginable, alone or in combination with other information sources.

In an embodiment of the injection system according to the present disclosure, a mode selection switch is provided on housing 1. The mode selection switch is used to switch between different injection modes. For example, between intermittent and continuous injection. It would also be possible to provide different sub-modes, i.e. intermittent injection with different speeds and continuous injection with different speeds. Typically, a user scrolls between the different modes by repeatedly pushing the mode selection switch. The current mode can be indicated by means of LED-lights or similar. In this embodiment, the actuation means 8, 9 would typically be force independent, i.e. the injection speed will be the same regardless the pressure force thereon.

It would be possible to provide the injection device 100 with an RFID-reader capable of reading RFID-tags provided on the cartridges 2. These tags can provide information to the injection device 100 about the content, volume, date of manufacture, etc. of the cartridge 2 and its content. The information could be displayed on an LCD-screen provided on the housing 1 or presented to the user by haptics. It would also be possible to arrange a safety circuit within the device 100 to prevent the use of the device in cases were cartridges 2 with no or with defect RFID-tags or were the RFID-tags indicates that the content of the cartridge 2 is not intended for use with the injection device 100 in question or that the cartridge as such is incompatible with the injector. This greatly enhances safety of the patients.

The injection device 100 can be constructed to have the plunger rod travelling a predetermined distance when a switch 8, 9 is depressed. This means that a only predetermined amount of the liquid composition is expelled through the injection needle 10 each time a switch is actuated, independently from how long and how hard the switch 8, 9 is depressed. This facilitates for a user in situations where it is of a high importance that a maximum amount of the liquid composition is not exceeded. Another possibility is that the liquid composition is continuously expelled through the injection needle 10 as long as the switch 8, 9 is actuated.

The injection device according to the present disclosure is useful for injecting liquid compositions, in particular viscous liquid compositions, such as gel compositions. Although gels display behaviour similar to solids, they contain mostly liquid, e.g., water. Injection of certain liquid compositions, such as hydrogels of e.g., hyaluronic acid or similar polymers, having high viscosities and high elasticity, requires rather high forces, especially in combination with small needles 10. This might have the effect that small amounts of the liquid composition may drip from the needle 10 even after motion of the plunger is stopped. Liquid composition dripping on the patient's skin may cause irritation and discomfort and should be avoided. Obviously, the dripping is also a waste of liquid composition and should as such be avoided. In order to prevent such drooling of the liquid composition, it is suggested that not only will the motor stop upon release of the actuation means, but it will even retract the plunger rod a certain distance. Due to its elastic properties, plunger 7 will be slightly compressed when travelling forward through cartridge 2. When the motion of the plunger 7 comes to a stop, a decompression will occur, i.e. the plunger will expand slightly to assume its normal shape. In other known solutions, the plunger could only expand in a forward direction causing the gel composition to drip out of the needle 10. Further, it is possible that air gets entrapped within the cartridge/needle system. During forward movement of the plunger 7, this air will be compressed within cartridge 2 and similar to the above-mentioned problem with the elasticity of the plunger this could cause gel composition to drip out of needle 10 even though the plunger 7 has come to a stop within cartridge 2. With the construction according to the present disclosure, however, plunger 7 may instead expand resp. move backwardly without substantial resistance towards the retracted plunger rod, thus eliminating said drooling.

In another embodiment of the present disclosure, the injection device is arranged to be able to perform aspiration prior to injection. This is done in order to avoid injection into a blood vessel. When the needle has been inserted, the plunger is first retracted a short distance and if the needle has been inserted into a vessel, blood will be sucked up into the needle. If this is the case, you normally retract the needle and re-insert it at a different location. Injection into a vessel could cause necrosis. In order to be able to retract the plunger, a different connection between the plunger in the cartridge and the plunger rod is preferable. One imaginable connection is a self-locking snap-fit that snaps into place as the plunger rod approaches the plunger. The connection can then be unfastened for example by hand when the cartridge has been emptied but automatic or semi-automatic unfastening mechanisms are also conceivable.

In another embodiment of the injection system according to the present disclosure, the device can be pre-programmed to perform a priming sequence as soon as the device is switched on or as soon as a new cartridge is inserted into the housing. This is done in order to expel any air within the needle and the connection between cartridge and needle prior to treatment. Of course, a manual priming could also be done by a user by simply pushing one of the actuation means 8, 9.

Within the injection device 100, a drive mechanism comprising a motor and a power source is contained. The power source could comprise any type of rechargeable battery, such as, but not limited to, nickel cadmium (Ni—Cd), nickel metal hydride (NiMh), lithium ion (Li-ion) or lithium ion polymer (Li-ion polymer). Due to good its relatively high energy density and the fact that it can be provided in almost any desired shape, the Li-ion polymer is perhaps the best suited type for this application. The motor is preferably a DC motor such as a brushless or stepper dc-motor. Other types of motors are also conceivable. In order to be able to use the device for the injection of hydrogels of e.g., hyaluronic acid or similar polymers, or similar products, it is preferable that the motor is powerful enough that a force of up to about 100 N can be exerted on the plunger and thereby on the gel composition. The motor may be connected to the plunger rod by means of any of a number of different gear arrangements. For example the plunger rod may be provided with an external thread along at least a part of its length and the motor is connected to the plunger rod through this external thread. In this embodiment, the plunger rod will perform its rectilinear motion under rotation. Another possible solution involves a worm gear driven by the motor and a set of cogs provided on the plunger rod. Upon actuation of the motor, the worm gear will rotate and thereby cause a rectilinear motion by the plunger rod. It would also be possible to connect the motor with cogs provided on the plunger rod through one or more cog wheels, also known as rack and pinion gearing.

The cartridge used with the device will typically be provided with an injection needle 10 having a size between 21-32 G when used for injection of hyaluronic acid gels. The device could, however, be used in other fields of applications as well were injection needles of other sizes, such as 7-34 G, come into question.

FIGS. 2a, 2b, 2c and 2d show four possible positions in which the device 100 according to the present disclosure may be held. In FIG. 2a, a user holds the device 100 similar to how a pen is held. The switch 8 is arranged at such a position that the user can actuate it with her/his index finger. Since the rechargeable battery and to some extent also the motor can be arranged with a high degree freedom within the housing 1 of the device 100, the centre of gravity of the device can be located at a position which is favourable for a user. If the centre of gravity is located towards the rear end, i.e. near the switch 9, the device can be perceived as uncomfortable and strenuous when used over longer periods of time. This is a major advantage of the device according to the present disclosure over devices according to the known prior art.

FIG. 2b shows the device when held in a second position, similar to how a traditional syringe is held. Many medical practitioners, plastic surgeons and other users are used to operate a traditional syringe and actually prefer to hold the device 100 like this rather than in the pen-like position. The device according to the prior art does, however, not allow a user to do so. This is a further main advantage of the device 100 according to the present disclosure over the prior art. In this second position, the user can actuate the device 100 by means of switch 9 by her/his thumb. Protruding finger grips are provided to a user by plate 3 provided at the cartridge 2. Alternatively, finger grips can be arranged at the housing 1. This has the advantage that cartridges can be used which do not have plate 3 as well. However, it is preferred that if finger grips are provided at the housing 1 they should not prevent the use of cartridges 2 having a plate 3. The housing 1 should be designed such that the finger grips of the housing and the plate 3 lie adjacent to each other when a cartridge 2 is received in the housing 1.

FIG. 2c shows the device when held in a position similar to that of FIG. 2b. In this case instead of using his or her thumb, the user actuates the device 100 by his or her palm. This position is preferred by some users and is made possible by the device 100 according to the present disclosure.

FIG. 2d shows yet another possible position, similar how to for example a screw driver is held. This is also a position that users prefer in certain situations and that is made possible with the injection device 100 according to the present disclosure.

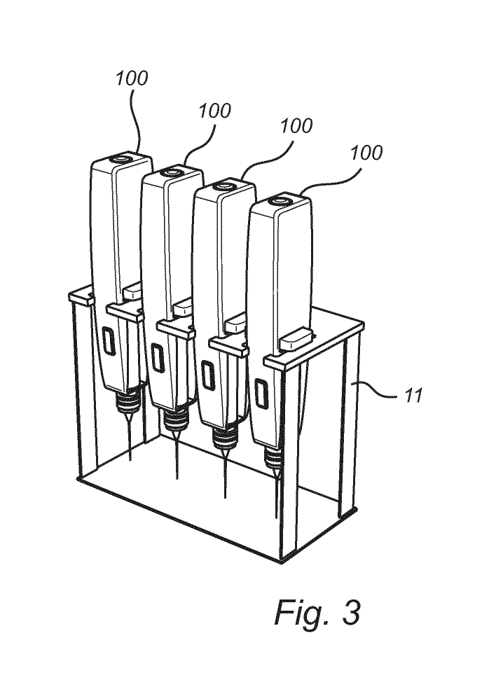

FIG. 3 schematically shows a plurality of devices 100 suspended in a combined charging and storing rack 11. The rack 11 may comprise a charging station which can charge the rechargeable batteries of the devices 100 by means of conductive or inductive charging. Typically, contact plates are provided on the housing 1 of each device 100 for connecting the devices 100 to the charging station. This type of connection has a number of advantages in this case. For example, no male/female cable connections for charging have to be provided on the housing 1 and the charging station respectively. This, in turn, has a plurality of benefits to it such as simplified cleaning, avoidance of defective contacts, and improved water impermeability. Furthermore, in order to charge the device, a user simply puts the device in the rack 11. It is not necessary to connect a power cable, or similar, to the device. It would also be possible to provide a rack having an internal re-chargeable battery which can be used to re-charge a plurality of injection devices before having to be re-charged itself. This provides for a great deal of flexibility for the user who can bring a freshly charged rack with one or more injection devices attached to it and bring to a treatment room and from that treatment room to others, depending on workplace structure, without having to worry about cords and the existence of power outlets in different rooms. The injection devices will then be re-charged also during transportation between treatment rooms. The internal battery of the rack will typically have such a capacity that a user does not have to re-charge the internal battery of the rack for at least one working day, preferably a plurality of days or even a whole week. In order to further enhance user-friendliness and mobility, the rack and the injection devices may be provided with corresponding snap connection elements such that the injection devices will be held firmly in place, e.g., during transportation thereof. The snap fit connection will also indicate to a user that the injection device is correctly inserted to the rack such that re-charging of the battery in the injection device certainly takes place.

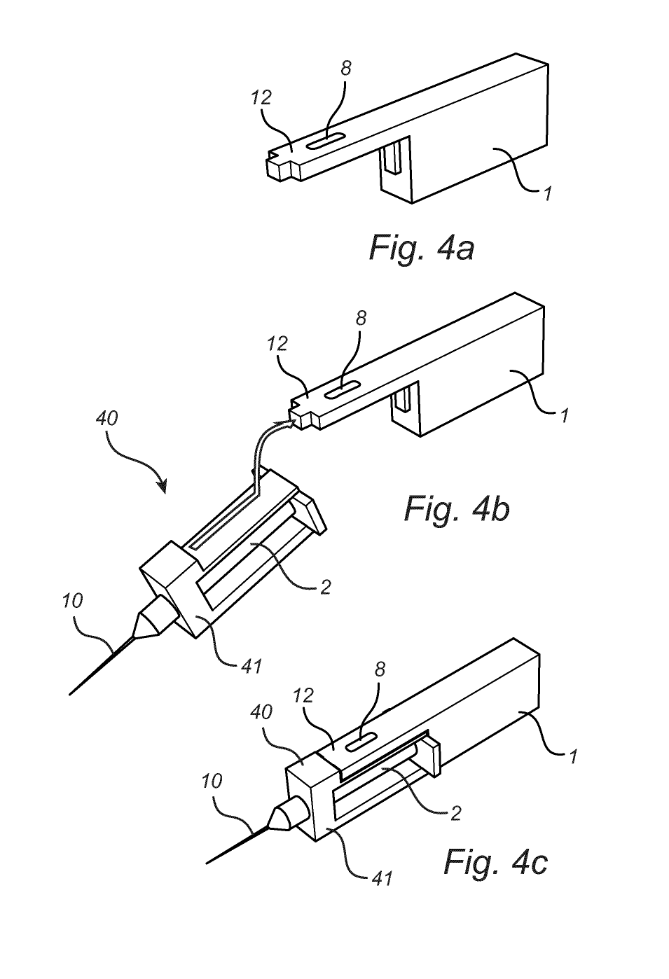

FIGS. 4a through 4c show another embodiment of the injection system according to the present disclosure. During use of the injection system it is the front part thereof that in most cases will exposed to any contaminations occurring during use. Therefore, it is advantageous to provide a large portion of the front of the injection system as a disposable part. Here, more or less the whole front of the system is made up of a disposable front piece 40 comprising a carrier 41 made from e.g., plastic. The carrier 41 can have a shape that corresponds and follows the shape of the housing 1 creating a homogeneous appearance with smooth transitions there between. Contained within the carrier 41 is a cartridge 2 having a needle 10 mounted thereon. Typically, carrier 41 and cartridge 2 are fixedly mounted to each other and should both be discarded once the cartridge 2 has been emptied. Housing 1 comprises a forwardly protruding element 12 on which actuation means 8 is arranged. This solution permits the construction of a housing 1 that has no, or at least very few, outer joints and all in all an outer casing that can be easily and reliably cleaned and disinfected between treatments. Front piece 40 is also arranged to protect the moveable parts of the re-usable housing 1 from contamination by shielding the exit of the plunger rod from the housing 1 and its entrance into the cartridge 2 towards the outside. Finger grips may also be provided on the carrier 41. The finger grips are also prone to contamination during use and it is therefore advantageous that they are a formed in the disposable part of the system.

In another embodiment of the injection system according to the present disclosure, it would also be possible to provide a housing having a generally planar front surface to which a disposable front piece can be coupled. In this case, the whole front part of the injection device would be constituted by a disposable front piece making cleaning and disinfecting even less of a problem since no part of the re-usable housing 1 would extend beyond the joint between the front piece and the housing 1.

Finally, it is realized, that an injection device according to the present disclosure has a number of advantages over the known prior art devices. Examples are a more appropriate centre of gravity, no need for time consuming and tedious pre-tensioning of springs and the fact that the device can be held in many different positions such as similar to how a pen is held and similar to a traditional syringe, using either the thumb or the palm of the hand for actuation of the device. This freedom of choice of holding position is enabled by the properties of the housing and the provision of actuating means on an upper side of the elongated housing, proximal to the first end of the housing, as well as at the second end of the housing. This allows a user to actuate the device either with her/his index finger when used in a pen position or with her/his thumb or palm of the hand when used as a traditional syringe or held as a screw driver (see FIG. 2d) respectively. The provision of all necessary equipment within the housing of the injection device, making it truly independent of any external equipment during use, makes the injection device according to the present disclosure exceptionally flexible and user friendly. No cords, external control units or external motors are required that could potentially hamper a user during use of the device. The injection of crosslinked or non-crosslinked hyaluronic acid gels has been mentioned as a possible area of use for the device according to the present disclosure. The hyaluronic acid gel is useful as a medical device, e.g., a dermal filler, for cosmetic use. It may also be useful in medical surgery, e.g., in eye surgery, joint surgery and medical cosmetic surgery or as a medicament, e.g., for treatment of joint disease. Naturally, it is possible to use the device according to the present disclosure with other liquid compositions, and preferably gel compositions, such as hydrogels. The device is also useful for injecting other types of dermal fillers than hyaluronic acid, e.g., collagen, calcium hydroxyl apatite, poly-L-lactic acid (PLLA), and polymethylmethacrylate (PMMA). Furthermore, the device is useful for injecting liquid compositions comprising active substances, e.g., bioactive agents, local anesthetics, cicatrizants, antioxidants or botulinum toxin. A preferred liquid composition of this type is a gel composition with a hyaluronic acid gel carrier and an active substance, e.g., a local anesthetic or a cictrizant, such as dextranomer beads.

1. An injection device for delivering a liquid composition, comprising:
an elongated housing having a first end and a second end;
said housing being configured to receive an exchangeable cartridge, comprising said liquid composition, the cartridge being received proximal to said first end of said housing; and
a drive mechanism arranged within said housing, said drive mechanism comprising an electric motor and a power source, wherein the electric motor is coupled to a plunger rod for acting on a cartridge received in said housing such that the liquid composition contained in the cartridge is expelled; and wherein a first actuation means for actuating the drive mechanism is provided on an upper side of the housing proximal to said first end of the housing,
wherein a second actuation means for actuating the drive mechanism is provided at said second end of the housing, and said first and second actuation means are arranged to actuate the drive mechanism independent from each other.

2. The injection device according to claim 1, wherein the power source comprises a rechargeable battery.

3. The injection device according to claim 2, wherein contact plates are provided on the housing through which the rechargeable battery is connected to a charging station for recharging.

4. The injection device according to claim 1, wherein finger grips are provided on opposite first and second side walls of the housing.

5. The injection device according to claim 4, wherein a position of the finger grips on said first and second side walls of the housing is adjustable.

6. The injection device according to claim 1, wherein the drive mechanism exerts a force of up to about 100 N on the liquid composition contained in a cartridge received in the housing.

7. The injection device according to claim 1, wherein said first and second actuation means comprise encapsulated switches.

8. The injection device according to claim 1, wherein at least one of the first and second actuation means comprises tactile feedback means for providing indications to a user about the status of an ongoing injection of the liquid composition.

9. The injection device according to claim 1, wherein at least one of the first and second actuation means comprises a touch switch.

10. The injection device according to claim 1, including means for obtaining and presenting information to a user pertaining to an inserted cartridge.

11. The injection device according to claim 1, wherein the housing is made from plastic.

12. The injection device according to claim 1, wherein at least a part of an outer surface of the housing is provided with a grip enhancing surface coating.

13. An injection system for delivering a liquid composition, comprising an injection device according to claim 1, an exchangeable cartridge containing said liquid composition, and an injection needle attached to said cartridge, wherein said cartridge is held by said housing.

14. The injection system for delivering a liquid composition according to claim 13, wherein the exchangeable cartridge comprises finger grips.

15. The injection system for delivering a liquid composition according to claim 13, wherein a diameter of the injection needle ranges from 7 to 32 gauge.

16. The injection system for delivering a liquid composition according to claim 13, wherein the liquid composition is a gel.
